(19) United States
(12) Patent Application Publication
(10) Pub. No.: US 2011/0123439 A1
(43) Pub. Date: May 26, 2011

(54) Dual-Modality PET/MRI Contrast Agents

(75) Inventors: Jin Woo Cheon, Seoul (KR); Jin-Sil Choi, Seoul (KR); Jeongsoo Yoo, Daegu (KR); Jeong Chan Park, Daegu (KR); Yongmin Chang, Daegu (KR)

(73) Assignees: Industry-Academic Cooperation Foundation, Yonsei University, Seoul (KR); Kyungpook National University Industry-Academic Cooperation Foundation, Daegu (KR)

(21) Appl. No.: 12/991,503

(22) PCT Filed: May 8, 2009
(86) PCT No.: PCT/KR2009/002441

§ 371 Date: Feb. 2, 2011

(30) Foreign Application Priority Data
May 9, 2008 (KR) 10-2008-0043665

(51) Int. Cl. A61K 51/12

(52) U.S. Cl. 424/1.37; 424/1.29

(57) ABSTRACT

The present invention relates a dual-modality PET (positron emission tomography)/MRI (magnetic resonance imaging) contrast agent, a hybrid nanoparticle comprising: (a) a magnetic signal generating core; (b) a water-soluble multi-functional ligand coated on the signal generating core; and (c) a positron emitting factor linked to the water-soluble multi-functional ligand. The contrast agent of the present invention is the dual-modality contrast agent enabling to perform PET and MR imaging and can effectively obtain images having the merits of PET (excellent sensitivity and high temporal resolution) and MR (high spatial resolution and anatomical information) imaging. The contrast agent of the present invention is very useful for non-invasive and highly sensitive real-time fault-free imaging of various biological events such as cell migration, diagnosis of various diseases (e.g., cancer diagnosis) and drug delivery.

### BACKGROUND OF THE INVENTION

1. Field of the Invention

The present invention relates to a dual-modality PET (positron emission tomography)/MRI (magnetic resonance imaging) contrast agent.

2. Description of the Related Art

As the imaging technique of biological targets becomes an increasingly important tool towards understanding of basic biological phenomena and fault-free diagnosis of various diseases, which needs to be excellent in the view of 1) sensitivity, 2) accuracy and 3) rapidity.

However, current single imaging modality methods tend to be not adequate, resulting in false diagnosis. Therefore, multi-modal imaging in which each single imaging modality method is combined rapidly becomes an essential tool in the art of imaging research and a standard practice in the clinic. By using dual- or triple-modality methods, many shortcomings that are present in single imaging modality methods can be overcome. For example, several combinations of different imaging modality such as PET/CT (computed tomography), MR (magnetic resonance)/optical and PET/NIRF (near infrared optical fluorescence) have already been attempted. Out of them, the dual-modality PET/MR imaging method enabling a non-invasive three-dimensional tomography retains advantages of each imaging tool: (a) as merits of PET, excellent sensitivity, high temporal resolution and biological functional imaging, and (b) as merits of MRI, high spatial resolution and in detail anatomical information. Accordingly, it is very likely to ensure fault-free diagnosis of various diseases and its applicability therefore becomes widened (S. S. Gambhir et al, Gene. Dev, 17: 545 (2003)). The dual-modality PET/MR imaging technique is disclosed in US Pat. Pub. Nos. US20060052685, US20080045829 and US20080033279. Recently, B. J. Pichler and his colleagues reported simultaneous PET-MRI equipment (Nature Medicine, 14: 459 (2008)).

In addition to the development of diagnosis devices, the development of multi-modal probe for improving accuracy and sensitivity of imaging techniques has been urgently demanded.

For high-sensitive and fault-free diagnosis of diseases in the dual-modality PET/MR imaging, dual-modality PET/MRI probes are required to possess the following features: 1) remarkable imaging ability on magnetic resonance imaging, 2) effective and stable linking of a positron emitting radioisotope with a MR signal generating core, 3) stable delivery and distribution into body, and 4) feasible binding with a biologically or chemically active substance.

Although the contrast agent for dual-modality PET/MR imaging has been reported, still it is in an early stage of development. Up to date, the dual-modality PET/MR imaging contrast agents have been developed as follows:

U.S. Pat. No. 5,928,958 discloses that a radioactive element is attached to an iron oxide and iron nanoparticle which is coated with polysaccharide or polyethylene glycol. US Pat. Pub. No. US2007025888 discloses a contrast agent having a core containing oxide, metal oxide or metal hydroxide, and a shell consisting of optically active material which includes radioactive isotope.

In addition, R. Weissleder research team in Harvard Medical School developed a trimodality contrast agent for atherosclerosis by linking a fluorescent and radioactive substance to a monodisperse iron oxide nanoparticle (MION) coated with dextran (Circulation, 117: 379 (2008)).

However, the techniques described above have some serious limitations:

In U.S. Pat. No. 5,928,958, the attachment process of additional radioactive isotopes is complicated and its efficiency is low since polysaccharide or polyethylene glycol coated on iron oxide and iron nanoparticle are composed of hydroxyl groups.

In core-shell contrast agent prepared by ion-exchange reaction suggested in US Pat. Pub. No. US2007025888, it is hard to obtain the equal signals in imaging because the fabrication method is complex and it is also difficult to prepare dual-modality PET/MRI contrast agent with homogeneous composition.

In trimodality contrast agent provided by R. Weissleder research team in Harvard University, the magnetic particles used are not homogeneous in size and have low crystallinity, being responsible for poor MR imaging potential. Therefore, the MR images obtained using these trimodality contrast agent played only accessory role in PET/CT imaging. In this regard, this technology is not considered to provide an effective multi-modality contrast agent.

As such, the development of the contrast agents for effective PET/MR imaging is still unsatisfactory. Therefore, it remains in the art to develop a novel dual-modality contrast agent for ensuring high imaging ability and complementary PET/MR imaging by stable linking of a PET signal generating factor and a MR generating factor.

Throughout this application, various publications and patents are referred and citations are provided in parentheses. The disclosures of these publications and patents in their entities are hereby incorporated by references into this application in order to fully describe this invention and the state of the art to which this invention pertains.

### SUMMARY OF THE INVENTION

It is an object of this invention to provide an effective dual-modality PET (positron emission tomography)/MRI (magnetic resonance imaging) contrast agent. Therefore, the present invention utilizes a magnetic signal generating core with excellent magnetic property and nuclear imaging effect, to which a positron emitting factor is effectively and stably attached. Consequently, the present invention provides the dual-modality PET/MRI contrast agent having remarkable imaging ability and highly accurate diagnosis.

Other objects and advantages of the present invention will become apparent from the following detailed description together with the appended claims and drawings.

### BRIEF DESCRIPTION OF THE DRAWINGS

FIG. 1 represents transmission electron microscopic (TEM) images of synthesized magnetic nanoparticle. Each a, b, c and d represents 15 nm sized Fe₃O₄, 15 nm sized MnFe₂O₄, 6 nm FePt and 15 nm Gd₂O₃, respectively. All particles exhibit a homogeneous size distribution (σ<10%).

FIG. 2 represents MnFe₂O₄ nanoparticles coated with several water-soluble multi-functional ligands.

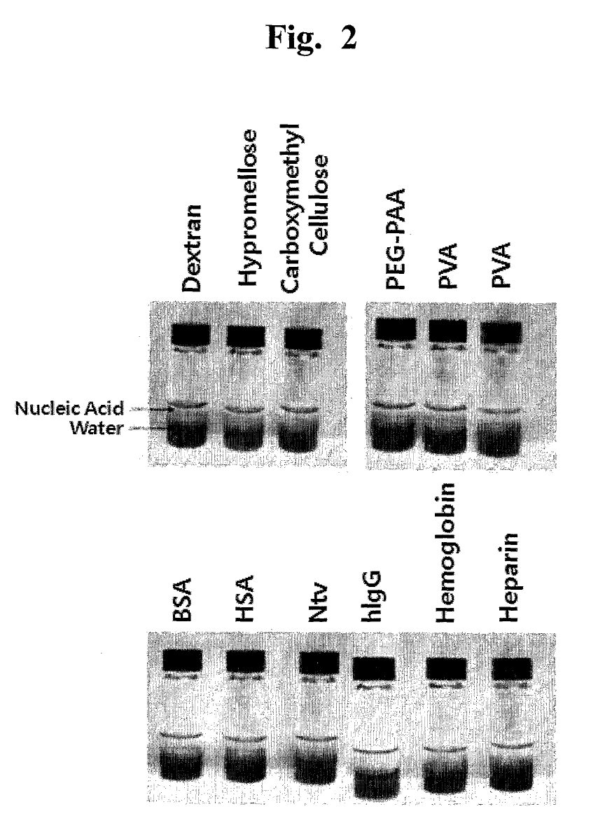

FIG. 3 represents the results measuring hydrodynamic size of MnFe₂O₄ coated with cross-linked serum albumins. FIG. 3a shows that the mean of hydrodynamic size is 32 nm as determined by dynamic light scattering. FIG. 3b represents retention time of nanoparticle in size exclusion column (Sepharcryl S-500, flow rate: 1 mL/min) as compared to that of various standard materials (thyroglobulin and ferritin), demonstrating a similar pattern with hydrodynamic size determined by dynamic light scattering. FIG. 3c represents cross-linked serum albumin (SA)-coated MnFe₂O₄ (SA-MnMEIO), which is stable in aqueous solution at various pH and salt concentrations (NaCl). It is clearly seen that nanoparticles are stable in aqueous solution at salt concentrations up to 1 M and wide pH range between 1-11.

FIG. 4 is a plot representing a magnetism of MnFe₂O₄. It is demonstrated that MnFe₂O₄ exhibits a superparamagnetic property and has a saturation magnetization (Ms) value of 124 emu/g (Mn+Fe).

FIG. 5 is a plot of T2 relaxivity coefficient (r₂) against Mn+Fe concentration for SA-MnFe₂O₄ (0.025, 0.050, 0.100, 0.200 mM (Mn+Fe)). T2 relaxivity coefficient (r₂) was measured to be 321.6 mM⁻¹s⁻¹.

FIG. 6 represents a radio-TLC measuring the labeling yield of ¹²⁴I-labelled SA-MnFe₂O₄. Each of region 1 and region 2 of FIG. 6 represents ¹²⁴I-labelled SA-MnFe₂O₄ and contaminants, respectively, and the labeling yield was in a range of not less than 90%.

FIG. 7 represents PET and MR images obtained from ¹²⁴I-labelled SA-MnFe₂O₄ (¹²⁴I-SA-MnFe₂O₄) diluted at various concentrations (200, 100, 50, 25, 12.5 μg/mL (Mn+Fe), 60, 30, 15, 7.5, 3.8 μCi/mL (124I)). It is demonstrated that PET and MR signals of ¹²⁴I-SA-MnFe₂O₄ are in accordance with MR signal of SA-MnFe₂O₄ and PET signal of free ¹²⁴I solutions diluted at equal concentrations.

FIG. 8 represents PET image from ¹²⁴I-SA-MnFe₂O₄ diluted at various radioactivities (20, 4, 0.8, 0.16, 0.032 μCi/mL (¹²⁴I)) suggesting that the contrast agent of the present invention exhibits PET signal sensitivity in a range of 0.8 to 4 μCi/mL (¹²⁴I).

FIG. 9 represents MR image of different tubings in which SA-MnFe₂O₄ solution of 50 mg/ml (Mn+Fe) is filled. Several tubes with an outer diameter of 1.6 mm and various inner diameters of 1 mm, 500, 250, 180 and 100 μm were arranged and fixed using 1% agarose. SA-MnFe₂O₄ solution containing Mn+Fe concentration (50 mg/mL) was filled in tubes and tertiary distilled water was filled in the tube with inner diameter of 1 mm as a control. MR images could be distinctly distinguished up to inner diameters of 0.25 mm of the tubes. However, MR signals could not be detected in a distinctly differentiated manner for the tubes with inner diameters of below 0.25 mm, due to detection limitations of MR device.

FIG. 10 is the result measuring PET and MR image in each model including ¹²⁴I-labelled SA-MnFe₂O₄, FePt and Fe₃O₄. Dual-modality synthetic probe exhibits an increase in PET and MR signal. The signals in tubes filled with each ¹²⁴I-labelled SA-MnFe₂O₄ (b), FePt (c) and Fe₃O₄ (d) solution in PET imaging were highly increased as compared with those of water (a). In addition, the signals in tubes filled with each ¹²⁴I-labelled SA-MnFe₂O₄ (f), FePt (g) and Fe₃O₄ (h) solution in MR imaging were significantly increased as compared with those of water (e).

FIG. 11 represents PET/MR images of sentinel lymph node (SLN) in a rat at 1 hr post-injection of ¹²⁴I-labelled SA-MnFe₂O₄ onto the right forepaw. In coronal MR (FIG. 11a) and PET (FIG. 11b) images, a brachial lymph node (brachial LN, white circle) is detected. In FIG. 11c, the position of the brachial LN is well matched in a PET/MR fusion image. In the transverse images of MRI (FIG. 11d) and PET (FIG. 11e), two lymph nodes, axillary (red circle) and brachial LNs (white circle), are detected and also completely overlap in the combined image (FIG. 11f).

FIG. 12 shows PET and MR images of the excised brachial LN of rat right after in vivo PET/MR imaging depicted in FIG. 11. The brachial LN was explanted and immobilized into 1% agarose gel. Only the LN from the right side of the rat containing ¹²⁴I-SA-MnMEIO shows strong PET and MR signals and the ex vivo experiments also show consistent results with in vivo images as shown in FIG. 11.

### DETAILED DESCRIPTION OF THIS INVENTION

In one aspect of this invention, there is provided a dual-modality PET (positron emission tomography)/MRI (magnetic resonance imaging) contrast agent, which comprises a hybrid nanoparticle comprising: (a) a magnetic signal generating core; (b) a water-soluble multi-functional ligand coated on the signal generating core; and (c) a positron emitting factor linked to the water-soluble multi-functional ligand.

The present inventors have carried out intensive studies to develop a dual-modality contrast agent for PET and MR imaging. As a result, we have discovered that the magnetic nanoparticle coated with the water-soluble multi-functional ligand having excellent magnetic property and MR imaging effect is linked to the positron emitting factor, providing the dual-modality contrast agent with imaging potentials of PET and MR.

PET and MRI have merits such as non-invasive imaging and three-dimensional tomography compared to other imaging techniques and can be widely applied for effective diagnosis and biological imaging technique. Therefore, two imaging techniques are combined into a single system such that the dual-modality PET/MRI is prepared as an ideal imaging modality which has not only high signal sensitivity but also excellent temporal and spatial resolution. For effective realization of this purpose, it is also essential to use the dual-modal contrast agent which enhances the imaging effect.

The present invention performs PET/MR imaging using a single contrast agent, obtaining both PET and MR images of desired biological tissues and/or organs.

The dual-modality PET/MRI contrast agent of this invention has the magnetic signal generating core for MR imaging. The term “magnetic signal generating core” refers to a magnetic nanoparticle which includes any one of paramagnetic or superparamagnetic nanoparticles used for MRI in the art.

According to a preferable embodiment, the magnetic signal generating core includes a metal, a metal chalcogen (Group 16 element), a metal pnicogen (Group 15 element), an alloy and a multi-component hybrid structure thereof.

According to a preferable embodiment, the metal nanoparticle used in the magnetic signal generating core includes transition metal elements, Lanthanide metals and Actinide metals. More preferably, the metal nanoparticle used in the signal generating core is selected from transition metal elements selected from the group consisting of Co, Mn, Fe and Ni, and Lanthanide metal elements and Actinide metal elements selected from the group consisting of Nd, Gd, Tb, Dy, Ho, Er and Sm, and the multi-component hybrid structure thereof.

Preferably, the metal chalcogen nanoparticle includes a M^{a}ₓA_{y}, M^{a}ₓM^{b}_{y}A_{z} nanoparticle (M^{a} and M^{b} independently represent one or more elements selected from Group 1 metal elements, Group 2 metal elements, transition metal elements, metal and metalloid elements of Group 13-15 elements, Lanthanide metal elements and Actinide metal elements; A is selected from the group consisting of O, S, Se, Te and Po; 0≦x≦32, 0≦y≦32, 0<z≦8) and the multi-component hybrid structure thereof.

More preferably, the metal chalcogen nanoparticle includes the M^{a}ₓA_{y}, M^{a}ₓM^{b}_{y}A_{z} nanoparticles (M^{a}=one or more elements selected from transition metal elements selected from the group consisting of Ba, Cr, Mn, Fe, Co, Ni, Cu, Zn, Cd, Hg, Nb, Mo, Zr, W, Pd, Ag, Pt and Au, Group 13-15 metal elements selected from the group consisting of Ga, In, Sn, Pb and Bi, and Lanthanide metal elements and Actinide metal elements selected from the group consisting of Gd, Tb, Dy, Ho, Er, Sm and Nd; M^{b}=one or more elements selected from the group consisting of Group 1 metal elements, Group 2 metal elements, transition metal elements, metal and metalloid elements of Group 13-15 elements, Lanthanide metal elements and Actinide metal elements; A is selected from the group consisting of O, S, Se, Te and Po; 0≦x≦32, 0≦y≦32, 0<z≦8), and the multi-component hybrid structure thereof.

Much more preferably, the metal chalcogen nanoparticle includes a M^{a}ₓO_{z}, M^{a}ₓM^{b}_{y}O_{z} nanoparticle [M^{a}=one or more elements selected from the group consisting of transition metal elements selected from the group consisting of Ba, Cr, Mn, Fe, Co, Ni, Cu, Zn, Cd, Hg, Nb, Mo, Zr, W, Pd, Ag, Pt and Au, and Lanthanide metal elements and Actinide metal elements selected from the group consisting of Gd, Tb, Dy, Ho, Er, Sm and Nd; M^{b}=one or more elements selected from the group consisting of Group 1 metal elements (Li or Na), Group 2 metal elements (Be, Ca, Mg, Sr, Ba or Ra), Group 13 elements (Ga or In), Group 14 elements (Si or Ge), Group 15 elements (As, Sb or Bi), Group 16 elements (S, Se or Te), transition metal elements (Sr, Ti, V, Cu, Y, Zr, Nb, Mo, Tc, Ru, Rh, Pd, Ag, Cd, Hf, Ta, W, Re, Os, Ir, Pt, Au or Hg), Lanthanide metal elements and Actinide metal elements (La, Ce, Pr, Nd, Pm, Sm, Eu, Gd, Tb, Dy, Ho, Er, Tm or Yb); 0≦x≦16, 0≦y≦16, 0<z≦8], and the multi-component hybrid structure thereof.

Most preferably, the metal oxide nanoparticle used in the signal generating core includes M′FeₓO_{y} (M′=Mn, Fe, Co or Ni, 0<x≦8, 0≦y≦8), Zn_{w}M″ₓFe_{y}O_{z} (0<w≦8, 0≦x≦8, 0<y≦8, 0<z≦8; M″ represents one or more metal atoms selected from the group consisting of Group 1 elements, Group 2 elements, Group 13 elements, transition metal elements, Lanthanide metal elements and Actinide metal elements) and M′″ₓO_{y} (M′″=Gd, Tb, Dy, Ho or Er, 0<x≦8, 0≦y≦16) nanoparticle.

The metal pnicogen nanoparticle preferably includes a M^{c}ₓA_{y}, M^{c}ₓM^{d}_{y}A_{z} nanoparticle (M^{c} and M^{d} independently represent the element selected from the group consisting of Group 1 metal elements, Group 2 metal elements, transition metal elements, metal and metalloid elements of Group 13-14 elements, Lanthanide metal elements and Actinide metal elements; A is selected from the group consisting of N, P, As, Sb and Bi; 0≦x≦40, 0≦y≦40, 0<z≦8), and the multi-component hybrid structure thereof.

More preferably, the metal pnicogen nanoparticle includes the M^{c}ₓA_{y}, M^{c}ₓM^{d}A_{z} nanoparticle (M^{c} represents the element selected from transition metal elements selected from the group consisting of Ba, Cr, Mn, Fe, Co, Ni, Cu, Zn, Cd, Hg, Nb, Mo, Zr, W, Pd, Ag, Pt and Au, Group 13-14 elements selected from the group consisting of Ga, In, Sn and Pb, and Lanthanide metal elements and Actinide metal elements selected from the group consisting of Gd, Tb, Dy, Ho, Er, Sm and Nd; M^{d}=one or more elements selected from the group consisting of Group 1 metal elements, Group 2 metal elements, transition metal elements, metal and metalloid elements of Group 13-14 elements, and Lanthanide metal elements and Actinide metal elements; A is selected from N, P, As, Sb and Bi; 0<x≦40, 0<y≦40, 0<z≦8), and the multi-component hybrid structure thereof.

The alloy nanoparticle includes a M^{e}ₓM^{f}_{y}, M^{e}ₓM^{f}_{y}M^{g}_{z} nanoparticle (M^{e}=one or more elements selected from transition metal elements selected from the group consisting of Ba, Cr, Mn, Fe, Co, Ni, Cu, Zn, Nb, Mo, Zr, Te, W, Pd, Ag, Pt and Au, and Lanthanide metal elements and Actinide metal elements selected from the group consisting of Gd, Tb, Dy, Ho, Er, Sm and Nd; M^{f} and Mg independently represent one or more elements selected from the group consisting of Group 1 metal elements, Group 2 metal elements, Group 13 elements, Group 14 elements, Group 15 elements, Group 16 elements, transition metal elements, Lanthanide metal elements and Actinide metal elements; 0<x≦20, 0<y≦20, 0<z≦20). Preferably, the alloy nanoparticle includes the M^{e}ₓM^{f}_{y} nanoparticle (M^{e} and M^{f} independently represent one or more elements selected from the group consisting of Co, Fe, Mn, Ni, Mo, Si, Al, Cu, Pt, Sm, B, Bi, Cu, Sn, Sb, Ga, Ge, Pd and In; 0<x≦20, 0≦y≦20).

According to a preferable embodiment, the magnetic signal generating core includes:

1) the metal nanoparticle, M (M=Ba, Cr, Mn, Fe, Co, Zn, Nb, Mo, Zr, Te, W, Pd, Gd, Tb, Dy, Ho, Er, Sm or Nd),

2) the alloy nanoparticle, M^{f}ₓM^{g}_{y} (M^{f} and M^{g} independently represent one or more elements selected from the group consisting of Co, Fe, Mn, Ni, Mo, Si, Al, Cu, Pt, Sm, B, Bi, Cu, Sn, Sb, Ga, Ge, Pd, In, Au, Ag and Y; 0<x≦20, 0≦y≦20),

3) the metal oxide nanoparticle, M^{a}ₓO_{y}, in the metal chalcogen nanoparticle (M^{a}=one or more elements selected from the group consisting of Ba, Cr, Co, Fe, Mn, Ni, Cu, Zn, Nb, Pd, Ag, Au, Mo, Si, Al, Pt, Sm, B, Bi, Sn, Sb, Ga, Ge, Pd, In, Gd, Tb, Dy, Ho, Er, Sm and Nd; 0<x≦16, 0≦y≦8),

and the multi-component hybrid structure thereof.

Most preferably, the inorganic nanoparticle core includes M^{h}ₓFe_{y}O_{z} (M^{h}=one or more elements selected from the group consisting of Ba, Mn, Fe, Co, Ni and Zn; 0≦x≦16, 0<y≦16, 0<z≦8) or Zn_{w}MⁱₓFe_{y}O_{z} (0<w≦16, 0≦x≦16, 0<y≦16, 0<z≦8; Mⁱ represents one or more elements selected from the group consisting of Group 1 metal elements, Group 2 metal elements, Group 13 metal elements, transition metal elements, Lanthanide metal elements and Actinide metal elements) nanoparticle core.

The multi-component hybrid structure includes two or more nanoparticles selected from the group consisting of metal, alloy, metal chalcogen or metal pnicogen nanoparticles described above, or one or more nanoparticles including both (i) the nanoparticle selected from the group consisting of metal, alloy, metal chalcogen or metal pnicogen nanoparticles described above and (ii) the nanoparticle selected from the group consisting of other metals (e.g., Au, Pt, Pd, Ag, Rh, Ru, Os or Ir), metal chalcogen and metal pnicogen. The multi-component hybrid structure has a core-shell, a multi-core shell, a heterodimer, a trimer, a multimer, a barcode or a co-axial rod structure.

The signal generating core exhibits remarkable imaging ability in MRI as magnetic property (magnetism) increases (S. H. Koenig et al. Magn. Reson. Med. 34: 227 (1995)). According to a preferable embodiment, the magnetic signal generating core in the contrast agent of this invention has a saturation magnetization (Mₛ) value of above 20 emu/g (magnetic element) and more preferably 50-1000 emu/g (magnetic element). According to a preferable embodiment, the signal generating core in the contrast agent of this invention has the spin relaxivity coefficient value (r₂) of above 50 mM⁻¹sec⁻¹, more preferably 100-3000 mM⁻¹sec⁻¹ and most preferably 150-1000 mM⁻¹sec⁻¹.

The contrast agent of this invention has to be stably dispersed in aqueous solution since it is finally administrated into animal, preferably human. For water-solubility, the contrast agent of this invention which includes the magnetic signal generating core is coated with a water-soluble multi-functional ligand. This multi-functional ligand to allow solubility in water may be any one used ordinarily in the art.

According to a preferable embodiment, the water-soluble multi-functional ligand comprises (i) an attachment region (L_{I}) to be linked to the signal generating core, and more preferably (ii) an active ingredient-binding region (L_{II}) for bonding of active ingredients, or (iii) a cross-linking region (L_{III}) for cross-linking between water-soluble multi-functional ligands, or (iv) a region which includes both the active ingredient-binding region (L_{II}) and the cross-linking region (L_{III}).

The term “attachment region (L_{I})” refers to a portion of the water-soluble multi-functional ligand including a functional group capable of binding to the magnetic signal generating core, and preferably to an end portion of the functional group. Accordingly, it is preferable that the attachment region including the functional group should have high affinity with the materials constituting the magnetic signal generating core. The magnetic signal generating core can be attached to the attachment region by an ionic bond, a covalent bond, a hydrogen bond, a hydrophobic interaction or a metal-ligand coordination bond. The attachment region of water-soluble multi-functional ligand may be varied depending on the substances constituting the magnetic signal generating core. For example, the attachment region (L_{I}) using ionic bond, covalent bond, hydrogen bond or metal-ligand coordination bond may include —COOH, —NH₂, —SH, —CONH₂, —PO₃H, —OPO₃H₂, —SO₃H, —OSO₃H, —N₃, —NR₃OH (R=CₙH₂ₙ₊₁, 0≦n≦16), —OH, —SS—, —NO₂, —CHO, —COX (X=F, Cl, Br or I), —COOCO—, —CONH— and —CN, and the attachment region (L_{I}) using hydrophobic interaction may include a hydrocarbon chain having two or more carbon atoms, but not limited to.

The term “active ingredient-binding region (L_{II})” means a portion of water-soluble multi-functional ligand containing the functional group capable of binding to chemical or biological functional substances, and preferably the other end portion located at the opposite side from the attachment region. The functional group of the active ingredient-binding region may be varied depending on the type of active ingredient and their formulae (Table 1). The active ingredient-binding region in this invention includes, but not limited to, —SH, —COOH, —CHO, —NH₂, —OH, —PO₃H, —OPO₃H₂, —SO₃H, —OSO₃H, —NR₃⁺X⁻ (R=CₙHₘ, 0≦n≦16, 0≦m≦34, X=OH, Cl or Br), NR₄⁺X⁻ (R=CₙHₘ, 0≦n≦16, 0≦m≦34, X=OH, Cl or Br), —N₃, —SCOCH₃, —SCN, —NCS, —NCO, —CN, —F, —Cl, —Br, —I, an epoxy group, —ONO₂, —PO(OH)₂, —C═NNH₂, —HC═CH— and —C≡C—.

TABLE 1

| I | II | III |
|---|---|---|
| R—NH₂ | R′—COOH | R—NHCO—R′ |
| R—SH | R′—SH | R—SS—R′ |
| R—OH | R′-(Epoxide group) | R—OCH₂CH(OH)—R′ |
| R—NH₂ | R′-(Epoxide group) | R—NHCH₂CH(OH)—R′ |
| R—SH | R′-(Epoxide group) | R—SCH₂CH(OH)—R′ |
| R—NH₂ | R′—COH | R—N═CH—R′ |
| R—NH₂ | R′—NCO | R—NHCONH—R′ |
| R—NH₂ | R′—NCS | R—NHCSNH—R′ |
| R—SH | R′—COCH₃ | R—COCH₂S—R′ |
| R—SH | R′—O(C═O)X | R—S(C═O)O—R′ |
| R-(Aziridine group) | R′—SH | R—CH₂CH(NH₂)CH₂S—R′ |
| R—CH═CH₂ | R′—SH | R—CH₂CH₂S—R′ |
| R—OH | R′—NCO | R—NHCOO—R′ |
| R—SH | R′—COCH₂X | R—SCH₂CO—R′ |
| R—NH₂ | R′—CON₃ | R—NHCO—R′ |
| R—COOH | R′—COOH | R—(C═O)O(C═O—R′) + H₂O |
| R—SH | R′—X | R—S-R′ |
| R—NH₂ | R′CH₂C(NH²⁺)OCH₃ | R—NHC(NH²⁺)CH₂—R′ |
| R—OP(O²⁻)OH | R′—NH₂ | R—OP(O²⁻)—NH—R′ |
| R—CONHNH₂ | R′—COH | R—CONHN═CH—R′ |
| R—NH₂ | R′—SH | R—NHCO(CH₂)₂SS—R′ |

(I: functional group of active ingredient-binding region in multi-functional ligand, II: active ingredient, III: exemplary bonds by reaction of I and II)

The term “cross-linking region (L_{III})” refers to a portion of the multi-functional ligand including the functional group capable of cross-linking to an adjacent water-soluble multi-functional ligand, and preferably a side chain attached to a central portion. The term “cross-linking” means that the multi-functional ligand is bound to another multi-functional ligand by intermolecular interaction or the multi-functional ligands are bound to each other by a molecular linker. The intermolecular interaction includes, but not limited to, hydrogen bond, covalent bond (e.g., disulfide bond) and ionic bond. Therefore, the cross-linkable functional group may be selected according to the kind of the intermolecular interaction. For example, the cross-linking region may include —SH, —COOH, —CHO, —NH₂, —OH, —PO₃H, —OPO₃H₂, —SO₃H, —OSO₃H, Si—OH, Si(MeO)₃, —NR₃⁺X⁻ (R=CₙHₘ, 0≦n≦16, 0≦m≦34, X=OH, Cl or Br), NR₄⁺X⁻ (R=CₙHₘ, 0≦n≦16, 0≦m≦34, X=OH, Cl or Br), —N₃, —SCOCH₃, —SCN, —NCS, —NCO, —CN, —F, —Cl, —Br, —I, an epoxy group, —ONO₂, —PO(OH)₂, —C═NNH₂, —HC═CH— and —C≡C— as the functional ligand.

The preferable multi-functional ligand of the present invention includes a chemical monomer, a polymer, a protein, a carbohydrate, a peptide, a nucleic acid, a lipid and an amphiphilic ligand.

Another preferable example of the water-soluble multi-functional ligand in the contrast agent of the present invention is a monomer which contains the functional group described above, and preferably dimercaptosuccinic acid since it originally contains the attachment region, the cross-linking region and the active ingredient-binding region. That is, —COOH on one side of dimercaptosuccinic acid is bound to the magnetic signal generating core and —COOH and —SH on the other end portion functions to bind to an active ingredient. In addition, —SH of dimercaptosuccinic acid acts as the cross-linking region by disulfide bond with another —SH. In addition to the dimercaptosuccinic add, other compounds having —COOH as the functional group of the attachment region and —COOH, —NH₂ or —SH as the functional group of the active ingredient-binding region may be utilized as the preferable multi-functional ligand.

Still another example of the preferable water-soluble multi-functional ligand in the contrast agent of the present invention includes, but not limited to, one or more polymer selected from the group consisting of polyphosphagen, polylactide, polylactide-co-glycolide, polycaprolactone, polyanhydride, polymaleic acid, a derivative of polymaleic acid, polyalkylcyanoacrylate, polyhydroxybutylate, polycarbonate, polyorthoester, polyethylene glycol, poly-L-lysine, polyglycolide, polymethyl methacrylate and polyvinylpyrrolidone.

Still another example of the preferable water-soluble multi-functional ligand in the contrast agent of the present invention is a peptide. Peptide is oligomer/polymer consisting of several amino acids. Since the amino acids have —COOH and —NH₂ functional groups in both ends thereof, peptides naturally have the attachment region and the active ingredient-binding region. In addition, the peptide that contains one or more amino acids having at least one of —SH, —COOH, —NH₂ and —OH as the side chain may be utilized as the preferable water-soluble multi-functional ligand. Particularly, the peptide including tyrosine may be used in bonding of the magnetic signal generating core and the positron emitting factor without further molecular linker.

In the water-soluble nanoparticles according to the present invention, still another example of the preferable multi-functional ligand is a protein. Protein is a polymer composed of more amino acids than peptides, that is, composed of several hundreds to several hundred thousands of amino acids. Proteins contains —COOH and —NH₂ functional group at both ends, and also contains a lot of —COOH, —NH₂, —SH, —OH, —CONH₂, and so on. Proteins may be used as the water-soluble multi-functional ligand because they naturally contain the attachment region, the cross-linking region and the active ingredient-binding region as described in peptide. In addition, protein containing numerous tyrosine residues may be effectively used in the conjugation of the magnetic signal generating core and the positron emitting factor. The preferable protein as the water-soluble multi-functional ligand is simple protein, complex protein, inducible protein or an analog thereof. Much more preferable example of the water-soluble multi-functional ligand includes, but not limited to, a hormone, a hormone analog, an enzyme, an enzyme inhibitor, a signal-transducing protein or its part, an antibody or its part, a light chain antibody, a binding protein or its binding domain, an antigen, an attachment protein, a structural protein, a regulatory protein, a toxic protein, a cytokine, a transcription factor, a blood coagulation factor and a plant defense-inducible protein. Most preferably, the water-soluble multi-functional ligand in the present invention includes, but not limited to, albumin, histone, protamine, prolamine, glutenin, antibody (immunoglobulin), antigen, avidin, cytochrome, casein, myosin, glycinin, carotene, hemoglobin, myoglobin, flavin, collagen, globular protein, light protein, streptavidin, protein A, protein G, protein S, lectin, selectin, angioprotein, anti-cancer protein, antibiotic protein, hormone antagonist protein, interleukin, interferon, growth factor protein, tumor necrosis factor protein, endotoxin protein, lymphotoxin protein, tissue plasminogen activator, urokinase, streptokinase, protease inhibitor, alkyl phosphocholine, surfactant, cardiovascular pharmaceutical protein, neuro pharmaceutical protein and gastrointestinal pharmaceuticals.

Still another example of the preferable water-soluble multi-functional ligand in the present invention is a nucleic acid. The nucleic acid is oligomer consisting of many nucleotides. Since the nucleic acids have PO₄⁻ and —OH functional groups in their both ends, they naturally have the attachment region and the active ingredient-binding region (L_{I}-L_{III}) or the attachment region and the cross-linking region (L_{I}-L_{II}). Therefore, the nucleic acids may be useful as the water-soluble multi-functional ligand in this invention. In some cases, the nucleic acid is preferably modified to have the functional group such as —SH, —NH₂, —COOH or —OH at 3′- or 5′-terminal ends.

Still another example of the preferable water-soluble multi-functional ligand in the contrast agent of the present invention is an amphiphilic ligand including both a hydrophobic and a hydrophilic region. In the nanoparticles synthesized in an organic solvent, hydrophobic ligands having long carbon chains coat the surface. When amphiphilic ligands are added to the nanoparticle solution, the hydrophobic region of the amphiphilic ligand and the hydrophobic ligand on the nanoparticles are bound to each other through intermolecular interaction to stabilize the nanoparticles. Further, the outermost part of the nanoparticles shows the hydrophilic functional group, and consequently water-soluble nanoparticles can be prepared. The intermolecular interaction includes a hydrophobic interaction, a hydrogen bond, a Van der Waals force, and so on. The portion which binds to the nanoparticles by the hydrophobic interaction is an attachment region (L_{I}), and further the amphiphilic cross-linking region (L_{II}) and the active ingredient-binding region (L_{III}) can be introduced therewith by an organo-chemical method. In addition, in order to increase the stability in an aqueous solution, amphiphilic polymer ligands with multiple hydrophobic and hydrophilic regions can be used. Cross-linking between the amphiphilic ligands can be also performed by a linker for enhancement of stability in an aqueous solution. Hydrophobic region of the amphiphilic ligand can be a linear or branched structure composed of chains containing 2 or more carbon atoms, more preferably an alkyl functional group such as ethyl, n-propyl, isopropyl, n-butyl, isobutyl, t-butyl, octyl, decyl, tetradecyl, hexadecyl, icosyl, tetracosyl, dodecyl, cyclopentyl and cyclohexyl; a functional group having an unsaturated carbon chain containing a carbon-carbon double bond, such as ethenyl, propenyl, isopropenyl, butenyl, isobutenyl, octenyl, decenyl and oleyl; and a functional group having an unsaturated carbon chain containing a carbon-carbon triple bond, such as propynyl, isopropynyl, butynyl, isobutynyl, octenyl and decenyl. In addition, examples of the hydrophilic region include the functional group being neutral at a specific pH, but being positively or negatively charged at a higher or lower pH such as —SH, —COOH, —NH₂, —OH, —PO₃H, —PO₄H₂, —SO₃H, —SO₄H and —NR₄⁺X⁻. Furthermore, preferable examples thereof include a polymer and a block copolymer, wherein monomers used include ethylglycol, acrylic acid, alkylacrylic acid, ataconic acid, maleic acid, fumaric acid, acrylamidomethylpropane sulfonic acid, vinylsulfonic acid, vinylphosphoric acid, vinyl lactic acid, styrenesulfonic acid, allylammonium, acrylonitrile, N-vinylpyrrolidone and N-vinylformamide, but not limited thereto.

Another example of the preferable water-soluble multi-functional ligand in the contrast agent of the present invention is a carbohydrate. More preferably, the carbohydrate includes, but not limited to, glucose, mannose, fucose, N-acetyl glucomine, N-acetyl galactosamine, N-acetylneuraminic acid, fructose, xylose, sorbitol, sucrose, maltose, glycoaldehyde, dihydroxyacetone, erythrose, erythrulose, arabinose, xylulose, lactose, trehalose, mellibose, cellobiose, raffinose, melezitose, maltoriose, starchyose, carrageenan, estrodose, xylan, araban, hexosan, fructan, galactan, mannan, agaropectin, alginic acid, hemicelluloses, hypromellose, amylose, deoxyacetone, glyceraldehyde, chitin, agarose, dextrin, ribose, ribulose, galactose, carboxy methylcellulose, glycogen dextran, carbodextran, polysaccharide, cyclodextran, pullulan, cellulose, starch and glycogen.

The compounds having the above-described functional group in nature may be used as the water-soluble multi-functional ligand. The compounds modified or prepared so as to have the above-described functional group according to a chemical reaction known in the art may be also used as the water-soluble multi-functional ligand.

According to a preferable embodiment, the water-soluble multi-functional ligand is cross-linked through cross-linking regions (L_{III}) or additional molecular linker. The cross-linking permits the water-soluble multi-functional ligand to be firmly coated on the signal generating core. In particular, it is advantageous in the senses that the contrast agent of the present invention is administrated into the body. For example, in the case using proteins as the water-soluble multi-functional ligand, protein coating may be significantly stabilized by cross-linking the carboxyl and amine group of proteins using N-(3-dimethylaminopropyl)-N-ethylcarbodiimide hydrochloride (EDC) and N-hydroxysulfosuccinimide (sulfo-NHS). Furthermore, protein coating may be remarkably stabilized by cross-linking between further molecular linker (2,2-ethylenedioxy bis-ethylamine) and the carboxyl group on the surface of protein using EDS and sulfo-NHS.

The term “positron emitting factor” in this invention includes any one of radioisotopes used in the art which release a positron (β⁺) to obtain PET image.

According to a preferable embodiment, the positron emitting radioisotope which is covalently bound to the water-soluble multi-functional ligand includes ¹⁰C, ¹¹C, ¹³O, ¹⁴O, ¹⁵O, ¹²N, ¹³N, ¹⁵F, ¹⁷F, ¹⁸F, ³²Cl, ³³Cl, ³⁴Cl, ⁴³Sc, ⁴⁴Sc, ⁴⁵Tl, ⁵¹Mn, ⁵²Mn, ⁵²Fe, ⁵³Fe, ⁵⁵Co, ⁵⁶Co, ⁵⁸Co, ⁶¹Cu, ⁶²Cu, ⁶²Zn, ⁶³Zn, ⁶⁴Cu, ⁶⁵Zn, ⁶⁶Ga, ⁶⁶Ge, ⁶⁷Ge, ⁶⁸Ga, ⁶⁹Ge, ⁶⁹As, ⁷⁰As, ⁷⁰Se, ⁷¹Se, ⁷¹As, ⁷²As, ⁷³Se, ⁷⁴Kr, ⁷⁴Br, ⁷⁵Br, ⁷⁶Br, ⁷⁷Br, ⁷⁷Kr, ⁷⁸Br, ⁷⁸Rb, ⁷⁹Rb, ⁷⁹Kr, ⁸¹Rb, ⁸²Rb, ⁸⁴Rb, ⁸⁴Zr, ⁸⁵Y, ⁸⁶Y, ⁸⁷Y, ⁸⁷Zr, ⁸⁸Y, ⁸⁹Zr, ⁹²Tc, ⁹³Tc, ⁹⁴Tc, ⁹⁵Tc, ⁹⁵Ru, ⁹⁵Rh, ⁹⁶Rh, ⁹⁷Rh, ⁹⁸Rh, ⁹⁹Rh, ¹⁰⁰Rh, ¹⁰¹Ag, ¹⁰²Ag, ¹⁰²Rh, ¹⁰³Ag, ¹⁰⁴Ag, ¹⁰⁵Ag, ¹⁰⁶Ag, ¹⁰⁸In, ¹⁰⁹In, ¹¹⁰In, ¹¹⁵Sb, ¹¹⁶Sb, ¹¹⁷Sb, ¹¹⁵Te, ¹¹⁶Te, ¹¹⁷Te, ¹¹⁷I, ¹¹⁸I, ¹¹⁸Xe, ¹¹⁹Xe, ¹¹⁹I, ¹¹⁹Te, ¹²⁰I, ¹²⁰Xe, ¹²¹Xe, ¹²¹I, ¹²²I, ¹²³Xe, ¹²⁴I, ¹²⁶I, ¹²⁸I, ¹²⁹La, ¹³⁰La, ¹³¹La, ¹³²La, ¹³³La, ¹³⁵La, ¹³⁶La, ¹⁴⁰Sm, ¹⁴¹Sm, ¹⁴²Sm, ¹⁴⁴Gd, ¹⁴⁵Gd, ¹⁴⁵Eu, ¹⁴⁶Gd, ¹⁴⁶Eu, ¹⁴⁷Eu, ¹⁴⁷Gd, ¹⁴⁸Eu, ¹⁵⁰Eu, ¹⁹⁰Au, ¹⁹¹Au, ¹⁹²Au, ¹⁹³Au, ¹⁹³Tl, ¹⁹⁴Au, ¹⁹⁵Tl, ¹⁹⁶Tl, ¹⁹⁷Tl, ¹⁹⁸Tl, ²⁰⁰Tl, ²⁰⁰Bi, ²⁰²Bi, ²⁰³Bi, ²⁰⁵Bi, ²⁰⁶Bi and derivatives thereof.

The positron emitting radioisotope may be directly linked to the active ingredient-binding region of the water-soluble multi-functional ligand or indirectly bound by using a linker. For example, ¹²⁴I may be directly linked to a benzene ring on a side chain of tyrosine residue of protein in the present invention using ¹²⁴I and a protein as a positron emitting radioisotope and the water-soluble multi-functional ligand, respectively.

In addition, various positron emitting radioisotopes may be bound to the water-soluble multi-functional ligand through a coordination bond by attachment of an additional chelating compound. Most preferably, the positron emitting radioisotope is linked to the water-soluble multi-functional ligand through the coordination bond by the attachment of a chelating compound such as DOTA (1,4,7,10-Tetraazacyclododecane-N,N′,N″,N′″-tetraacetic acid) and its derivatives, TETA (1,4,8,11-Tetraazacyclotetradecane-14,8,11-tetraacetic acid) and its derivatives, EDTA (Ethylene Di-amine Tetra-acetic Acid) and its derivatives, DTPA (Diethylene Triamine Pentaacetic Acid) and its derivatives, and so on.

In addition to imaging ability, the contrast agent of the present invention refers to a nanoparticle in which a biomolecule (example: an antibody, a protein, an antigen, a peptide, a nucleic acid, an enzyme, a cell, etc.) or a chemically active substance (example: a monomer, a polymer, an inorganic support, a fluorescent substance, a drug, etc.) are bound to the active ingredient of the ligand in dual-modality PET/MRI contrast agent through a covalent bond, an ionic bond or a hydrophobic interaction. Further example of the biomolecule includes, but not limited to, an antibody, a protein, an antigen, a peptide, a nucleic acid, an enzyme and a cell, and preferably a protein, a peptide, DNA, RNA, an antigen, hapten, avidin, streptavidin, neutravidin, protein A, protein G, lectin, selectin, hormone, interleukin, interferon, growth factor, tumor necrosis factor, endotoxin, lymphotoxin, urokinase, streptokinase, tissue plasminogen activator, hydrolase, oxido-reductase, lyase, biological active enzymes such as isomerase, synthetase, enzyme cofactor and enzyme inhibitor.

The chemically active substance includes several functional monomers, polymers, inorganic substances, fluorescent organic substances or drugs.

Exemplified monomer described herein above includes, but not limited to, a drug containing anti-cancer drug, antibiotics, Vitamins, folic acid, a fatty acid, a steroid, a hormone, a purine, a pyrimidine, a monosaccharide and a disaccharide. The side chain of the above-described monomer includes one or more functional groups selected from the group consisting of —COOH, —NH₂, —SH, —SS—, —CONH₂, —PO₃H, —OPO₄H₂, —PO₂(OR¹)(OR²) (R¹, R²=CₛHₜNᵤO_{w}SₓP_{y}X_{z}, X=—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —SO₃H, —OSO₃H, —NO₂, —CHO, —COSH, —COX, —COOCO—, —CORCO— (R=CₗHₘ, 0≦l≦3, 0≦m≦2l+1), —COOR, —CN, —N₃, —N₂, —NROH (R=CₛHₜNᵤO_{w}SₓP_{y}X_{z}, X=—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —NR¹NR²R³ (R¹, R², R³=CₛHₜNᵤO_{w}SₓP_{y}X_{z}, X=—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —CONHNR¹R² (R¹, R²=CₛHₜNᵤO_{w}SₓP_{y}X_{z}, X=—F, —Cl, —Br or —I, 0≦s≦20, 0≦t≦2(s+u)+1, 0≦u≦2s, 0≦w≦2s, 0≦x≦2s, 0≦y≦2s, 0≦z≦2s), —NR¹R²R³X′ (R¹, R², R³=CₛHₜNᵤO_{w}SₓP_{y}X_{z}, X=—F, —Cl, —Br or —I, X′=F⁻, Cl⁻, Br⁻ or I⁻, 0≦s≦20, 0≦t≦2(s+u)+1, 0≦u≦2s, 0≦w≦2s, 0≦x≦2s, 0≦y≦2s, 0≦z≦2s), —OH, —SCOCH₃, —F, —Cl, —Br, —I, —SCN, —NCO, —OCN, -epoxy, -hydrazone, -alkene and alkyne group.

The example of the above-described chemical polymer includes dextran, carbodextran, polysaccharide, cyclodextran, pullulan, cellulose, starch, glycogen, monosaccharides, disaccharides and oligosaccharides, polyphosphagen, polylactide, polylactide-co-glycolide, polycaprolactone, polyanhydride, polymaleic acid and a derivative of polymaleic acid, polyalkylcyanoacrylate, polyhydroxybutylate, polycarbonate, polyorthoester, polyethylene glycol, poly-L-lysine, polyglycolide, polymethyl methacrylate, polymethylether methacrylate and polyvinylpyrrolidone, but not limited to.

Exemplified chemical inorganic substance described above includes a metal oxide, a metal chalcogen compound, an inorganic ceramic material, a carbon material, a semiconductor substrate consisting of group II/VI elements, group III/VI elements and group IV elements, and a metal substrate or complex thereof, and preferably, SiO₂, TiO₂, ITO, nanotube, graphite, fullerene, CdS, CdSe, CdTe, ZnO, ZnS, ZnSe, ZnTe, Si, GaAs, AlAs, Au, Pt, Ag and Cu.

The example of the above-described chemical fluorescent substance includes fluorescein and its derivatives, rhodamine and its derivatives, lucifer yellow, B-phytoerythrin, 9-acridine isothiocyanate, lucifer yellow VS, 4-acetamido-4′-isothio-cyanatostilbene-2,2′-disulfonate, 7-diethylamino-3-(4′-isothiocyatophenyl)-4-methylcoumarin, succinimidyl-pyrenebutyrate, 4-acetamido-4′-isothio-cyanatostilbene-2,2′-disulfonate derivatives, LC™-Red 640, LC™-Red 705, Cy5, Cy5.5, resamine, isothiocyanate, diethyltriamine pentaacetate, 1-dimethylaminonaphthyl-5-sulfonate, 1-anilino-8-naphthalene, 2-p-toluidinyl-6-naphthalene, 3-phenyl-7-isocyanatocoumarin, 9-isothiocyanatoacridine, acridine orange, N-(p-(2-benzoxazolyl)phenyl)meleimide, benzoxadiazol, stilbene and pyrene, but not limited to.

All PET and MR images can be obtained using the contrast agent of this invention. This property allows the contrast agent of this invention to obtain all advantages of PET and MR imaging. Consequently, the images which are reflected not only excellent in sensitivity and high temporal resolution of PET but also in high spatial resolution of MRI can be simultaneously obtained.

The dual-modality PET/MRI contrast agent of the present invention exhibits very high stability. The term “stability” refers to a property that a contrast agent particle is homogeneously dispersed in a dispersion solvent for a long time. Preferably, the stability is maintained in a range of above ˜10 mM of salt concentration. Preferably, the contrast agent of the present invention is also stable in aqueous solution with ˜0.25 M salt concentration and pH range between 5-10. The excellent stability permits the contrast agent of this invention not only to significantly enhance the bioavailability but also to be very advantageous for the development and storage of products.

According to a preferable embodiment, the contrast agent of the present invention has a hydrodynamic size in a range of 2 nm-500 μm and more preferably 10 nm-50 μm.

The dual-modality PET/MRI contrast agent of the present invention is very useful in imaging an internal region of human body. The imaging procedure is as follows: 1) the diagnostically effective amount of contrast agent is administrated into human, and 2) the human body is scanned by PET and MR imaging to obtain an optical image of the internal region (tissue) of human body.

In particular, the dual-modality PET/MRI contrast agent is suitable for cancer imaging.

The dual-modality PET/MRI contrast agent of the present invention may be administrated together with a pharmaceutically acceptable carrier, which is commonly used in pharmaceutical formulations, but is not limited to, includes lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methylcellulose, methylhydroxy benzoate, propylhydroxy benzoate, talc, magnesium stearate, and mineral oils. Details of suitable pharmaceutically acceptable carriers and formulations can be found in Remington's Pharmaceutical Sciences (19th ed., 1995), which is incorporated herein by reference.

The contrast agent according to the present invention may be parenterally administered. In the case that the contrast agent is administered parenterally, it is preferably administered by intravenous, intramuscular, intra-articular, intra-synovial, intrathecal, intrahepatic, intralesional or intracranial injection.

A suitable dose of the contrast agent of the present invention may vary depending on pharmaceutical formulation methods, administration methods, the patient's age, body weight, sex, pathogenic state, diet, administration time, administration route, an excretion rate and sensitivity for a used contrast agent. The term “diagnostically effective amount” refers to an amount which is enough to show and accomplish PET and MR image of human body.

The method to obtain PET and MR image using the contrast agent of the present invention may be carried out according to a conventional method. For example, PET imaging methods and devices are disclosed in U.S. Pat. No. 6,151,377, No. 6,072,177, No. 5,900,636, No. 5,608,221, No. 5,532,489, No. 5,272,343 and No. 5,103,098, which are incorporated herein by reference. MR imaging method and devices are disclosed in D. M. Kean and M. A. Smith, Magnetic Resonance Imaging: Principles and Applications (William and Wilkins, Baltimore 1986), U.S. Pat. No. 6,151,377, No. 6,144,202, No. 6,128,522, No. 6,127,825, No. 6,121,775, No. 6,119,032, No. 6,115,446, No. 6,111,410 and No. 602,891, which are incorporated herein by reference.

The dual-modality PET/MRI contrast agent of the present invention may be applied to a wide variety of biological organs or tissues, preferably imaging of lymphatic system. More preferably, the dual-modality PET/MRI contrast agent is suitable for imaging of sentinel lymph node (SLN).

Interestingly, the contrast agent of this invention enables to perform successfully dual-imaging of SLN of which images have been hardly known to obtain. The lymphatic system has roles in a main defense mechanism against infections and a passage in metastasis of malignant tumor. Therefore, it is critical to exactly demonstrate local positions and features of SLNs in determination of cancer progression, surgical resection and treatment region.

The present invention provides a nanoparticle-based probe for accomplishing the dual-modality PET/MR imaging, which has an excellent colloidal stability and feasible binding ability. Using the dual-modality contrast agent of this invention, PET/MR fusion images for a variety of biological tissues and/or organs may be definitely obtained due to excellent complementary nature of PET/MR imaging techniques. The hybrid probe of the present invention is very useful for non-invasive and highly sensitive real-time imaging of various biological events such as cell migration, diagnosis of various diseases (e.g., cancer diagnosis) and drug delivery.

The dual-modality contrast agent of the present invention provides stable dual-modality PET/MR imaging information with superior-sensitivity and high-accuracy because the magnetic signal generating core and the positron emitting factor are linked to each other in the contrast agent in more effective and stable manner. In addition, the dual-modality contrast agent of the present invention is stable in aqueous solution, which is very useful for non-invasive and highly sensitive real-time imaging of various biological events such as cell migration, diagnosis of various diseases (e.g., cancer diagnosis) and drug delivery.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1

### Synthesis of Magnetic Nanoparticles

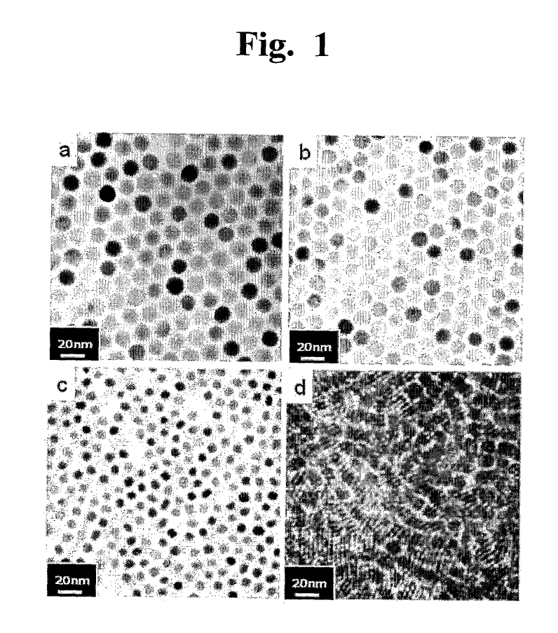

Fe₃O₄ and MnFe₂O₄ nanoparticles used in the experiments were synthesized according to the methods disclosed in Korean Pat. No. 0604975 and PCT/KR2004/003088. As precursors of nanoparticles, MCl₂ (M=Mn²⁺, Fe²⁺, Gd²⁺) (Aldrich, USA) and Fe(acac)₃ (Aldrich, USA), were added to trioctylamine solvent (Aldrich, USA) containing 4 mmol oleic acid (Aldrich, USA) and 4 mmol oleylamine (Aldrich, USA) as capping molecules. The mixture was incubated at 200° C. under argon gas atmosphere and further reacted at 300° C. The nanoparticles synthesized were precipitated by excess ethanol and then isolated. The isolated nanoparticles were again dispersed in toluene, generating a colloid solution. All synthetic nanoparticles exhibited a homogeneous size distribution (s<10%) (FIG. 1a, FIG. 1b and FIG. 1d).

FePt nanoparticles used in the experiments were synthesized according to the methods known to those skilled in the art (Shouheng Sun et al. Journal of the American Chemical Society, 126: 8394 (2004)). As precursors of nanoparticles, 1 mmol of Fe(CO)₅ (Aldrich, USA) and 0.5 mmol of Pt(acac)₂ (Aldrich, USA) were added to dioctylether solvent (Aldrich, USA) containing 2 mmol oleic acid (Aldrich, USA) and 2 mmol oleylamine (Aldrich, USA) as capping molecules. The mixture was incubated at 200° C. under argon gas and further reacted at 300° C. The nanoparticles synthesized were precipitated by excess ethanol and then isolated. The isolated nanoparticles were again dispersed in toluene, generating a colloid solution. All synthetic nanoparticles had an particle size of 6 nm with a homogeneous size distribution (s<10%) (FIG. 1c).

### Example 2

### Preparation of Serum Albumin-Coated Nanoparticles

Serum albumin (SA)-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of serum albumin (Aldrich, USA) was dissolved in 1 mL of deionized water and mixed with the precipitates, synthesizing nanoparticles coated with SA of rat. Finally, non-reactive SA was removed using a Sephacryl S-300 column (GE healthcare, USA), obtaining pure SA-coated water-soluble nanoparticles.

### Example 3

### Preparation of Immunoglobulin G-Coated Nanoparticles

Immunoglobulin G (IgG)-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of human IgG (hIgG) was dissolved in 1 mL of deionized water and mixed with the precipitates, synthesizing hIgG-coated nanoparticles. Finally, non-reactive hIgG was removed using a Sephacryl S-300 column, obtaining pure hIgG-coated water-soluble nanoparticles.

### Example 4

### Preparation of Neutravidin (Ntv)-Coated Nanoparticles

Ntv-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of Ntv was dissolved in 1 mL of deionized water and mixed with the precipitates, synthesizing Ntv-coated nanoparticles. Finally, non-reactive Ntv was removed using a Sephacryl S-300 column, obtaining pure Ntv-coated water-soluble nanoparticles.

### Example 5

### Preparation of Hemoglobin-Coated Nanoparticles

Hemoglobin-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of hemoglobin was dissolved in 1 mL of deionized water and mixed with the precipitates, synthesizing hemoglobin-coated nanoparticles. Finally, non-reactive hemoglobin was removed using a Sephacryl S-300 column, obtaining pure hemoglobin-coated water-soluble nanoparticles.

### Example 6

### Preparation of Heparin-Coated Nanoparticles

Heparin-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of heparin was dissolved in 1 mL of deionized water and mixed with the precipitates, synthesizing heparin-coated nanoparticles. Finally, non-reactive heparin was removed using a Sephacryl S-300 column, obtaining pure heparin-coated water-soluble nanoparticles.

### Example 7

### Preparation of Dextran-Coated Nanoparticles

Dextran-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of dextran was dissolved in 1 mL of deionized water and mixed with the precipitates, synthesizing dextran-coated nanoparticles. Finally, non-reactive dextran was removed using a Sephacryl S-300 column, obtaining pure dextran-coated water-soluble nanoparticles.

### Example 8

### Preparation of Hypromellose-Coated Nanoparticles

Hypromellose-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of hypromellose (M.W. 80,000) was dissolved in 1 mL of deionized water and mixed with the precipitates, synthesizing hypromellose-coated nanoparticles. Finally, non-reactive hypromellose was removed using a Sephacryl S-300 column, obtaining pure hypromellose-coated water-soluble nanoparticles.

### Example 9

### Preparation of Carboxymethylcellulose-Coated Nanoparticles

Carboxymethylcellulose-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of carboxymethylcellulose (M.W. 90,000) was dissolved in 1 mL of deionized water and mixed with the precipitates, synthesizing carboxymethylcellulose-coated nanoparticles. Finally, non-reactive carboxymethylcellulose was removed using a Sephacryl S-300 column, obtaining pure carboxymethylcellulose-coated water-soluble nanoparticles.

### Example 10

### Preparation of Polyvinylalcohol (PVA)-Coated Nanoparticle

PVA-coated nanoparticles were prepared according to the methods described in Korean Pat. No. 10-0604975, No. 10-0652251, No. 10-0713745, PCT/KR2004/002509 and PCT/KR2007/001001. Water-insoluble nanoparticles (5 mg) obtained were dispersed in 1 mL of 1 M NMe₄OH butanol solution and then homogeneously mixed for 5 min. Dark brown precipitates formed were separated by centrifugation (2,000 rpm, room temperature, 5 min). 10 mg of PVA (M.W. 10,000) was dissolved in 1 mL of deionized water and mixed with the precipitates, synthesizing PVA-coated nanoparticles. Finally, non-reactive PVA was removed using a Sephacryl S-300 column, obtaining pure PVA-coated water-soluble nanoparticles.

### Example 11

### Preparation of Polyethyleneglycol-Polyacrylate (PAA-PEG)-Coated Nanoparticles

PAA-PEG-coated nanoparticles were prepared according to the methods described in Korean Pat. No. 10-0604975, No. 10-0652251, No. 10-0713745, PCT/KR2004/002509 and PCT/KR2007/001001. PAA-PEG polymer was prepared in accordance with the following procedure. 0.72 g of PAA (M.W. 2,000) was dissolved in 10 mL of dichloromethane and mixed with 0.8 g of N-hydroxysuccinimide (NHS). 1.1 g of dicyclohexylcarbodiimide (DCC) was added to the mixture and incubated for 24 hrs. The resulting NHS-modified PAA was separated using a column chromatography and the solvent was removed, obtaining white solid materials. 0.8 g of the white solid material was dissolved in DMF solution and mixed with 2 g of NH₂-PEG-OH, followed by incubating for 24 hrs. Eventually, 50% PEG substituted PAA-PEG was yielded.

The water-insoluble nanoparticles (5 mg) were dispersed in ethanol solution (5 mg/mL) containing 1 mL of PAA-PEG and then homogeneously mixed for 10 hrs. Dark brown precipitates formed were separated by centrifugation (2,000 rpm, room temperature, 5 min). The precipitates were dissolved in 1 mL of deionized water and mixed with the precipitates, synthesizing PAA-PEG-coated nanoparticles. Non-reactive PAA-PEG was removed using a Sephacryl S-300 column, giving pure PAA-PEG-coated water-soluble nanoparticles.

### Example 12

### Preparation of Dimercaptosuccinate (DMSA)-Coated Nanoparticles

DMSA-coated nanoparticles were prepared according to the methods described in Korean Pat. No. 10-0604975, No. 10-0652251 and No. 10-0713745, PCT/KR2004/002509 and PCT/KR2007/001001. Water-insoluble nanoparticles (5 mg) obtained were dissolved in 1 mL of toluene solution. The mixture was mixed with 0.5 mL of methanol including 20 mg of 2,3-dimercaptosuccinate (DMSA). After reaction for 24 hrs, dark brown precipitates formed were separated by centrifugation (2,000 rpm, room temperature, 5 min) and was again dispersed in 1 mL of deionized water. The mixture was adjusted to pH 7-8 using 1 M NaOH, synthesizing DMSA-coated nanoparticles. Finally, non-reactive DMSA was removed using a Sephadex G-25 column, obtaining pure DMSA-coated water-soluble nanoparticles.

### Example 13

### Preparation of Cross-Linked Serum Albumin (SA)-Coated Nanoparticles

The nanoparticles were dispersed in 1 mL of 0.01 mol PBS buffer (pH 7.2), and N-(3-dimethylaminopropyl)-N-ethylcarbodiimide hydrochloride (50 μmol) and N-hydroxysulfosuccinimide (5 μmol) were added to the solution, followed by reacting for 2 hrs at room temperature. Cross-linked nanoparticles were purified by a DeSalting column (GE healthcare, USA).

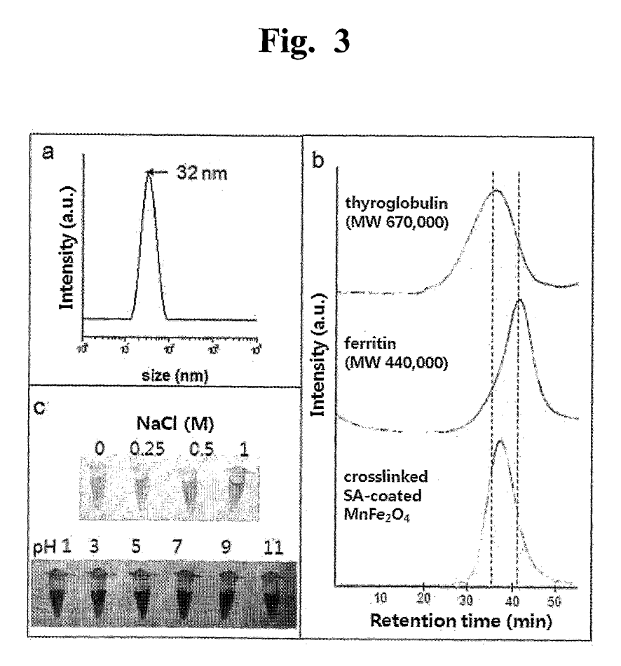

Hydrodynamic size of cross-linked SA-MnFe₂O₄ was measured to be 32 nm (FIGS. 3a-3b). SA-MnFe₂O₄ was stable in aqueous solution with salt concentration up to 1 M and wide pH range between 1-11 (FIG. 3c).

### Example 14

### Preparation of Cross-Linked Serum Albumin (SA)-Coated Nanoparticle 2

Nanoparticles were dispersed in 1 mL of 0.01 mol PBS buffer (pH 7.2), and 2,2-ethylenedioxy bis-ethyleneamine and N-(3-dimethylaminopropyl)-N-ethylcarbodiimide hydrochloride (50 μmol) and N-hydroxysulfosuccinimide (5 μmol) were added to the solution, followed by reacting for 2 hrs at room temperature. Cross-linked SA-MnFe₂O₄ was purified by a DeSalting column (GE healthcare, USA).

### Example 15

### Saturation Magnetization (Mₛ) Measurement of MnFe₂O₄

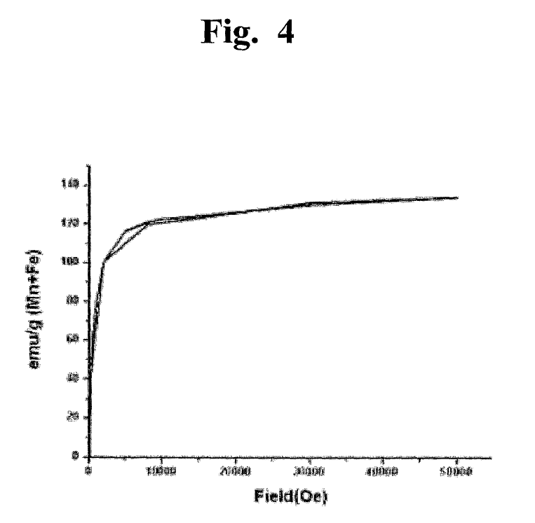

Synthesized MnFe₂O₄ and Gd₂O₃ were dried, producing their powders. Saturation Magnetization (Mₛ) was measured using a SQUID (Superconducting Quantum Interference Devices). MnFe₂O₄ exhibits a superparamagnetic property and has a saturation magnetization (Ms) value of 124 emu/g (Mn+Fe) (FIG. 4).

### Example 16

### T2 Relaxivity Coefficient (r2) Measurement of SA-MnFe₂O₄

The cross-linked SA-MnFe₂O₄ solutions were prepared in the concentrations of 0.1, 1, 10 and 100 μg (Mn+Fe)/mL. The T2 relaxivity coefficient (r2) was measured by using different echo time in a fast Spin Echo sequence (MRI equipment, repetition time (TR)=4000, echo time (TE)=10, 30, 60, 90, 120, 150, 180, 210, 240, 270, 300, 350, 400, 450, 500, 550, 600, 650, 700, 750, 800, 850, 900, 950, 1400, 1500, 1800, and 1900 ms, field of view (FOV)=9×9 cm, matrix=320×160, slice thickness=5 mm).

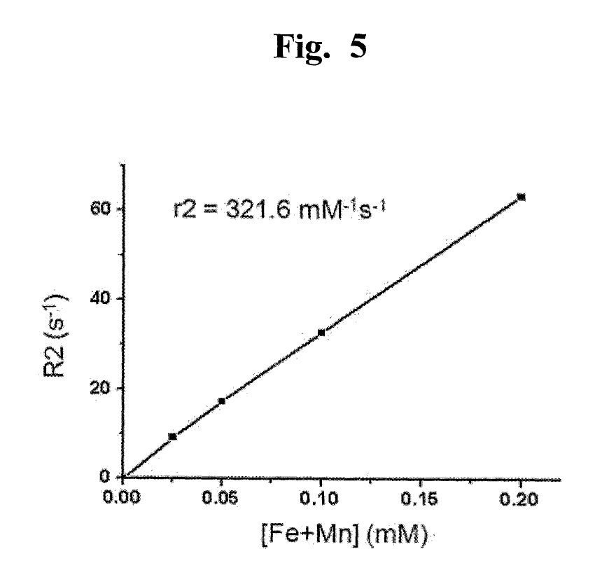

The T2 relaxivity coefficient (r₂) of SA-MnFe₂O₄ was measured to be 321.6 mM⁻¹s⁻¹ (FIG. 5), suggesting that SA-MnFe₂O₄ of the present invention enhances MR imaging effect. The T2 relaxivity coefficient (r2) of SA-MnFe₂O₄ is 2-3 folds higher than that of a conventional iron oxide-based SPIO (superparamagnetic iron oxide) probe (G. P. Krestin et al., Eur. Radiol., 11: 2319 (2001)).

### Example 17

### Labeling of SA-MnFe₂O₄ with ¹²⁴I

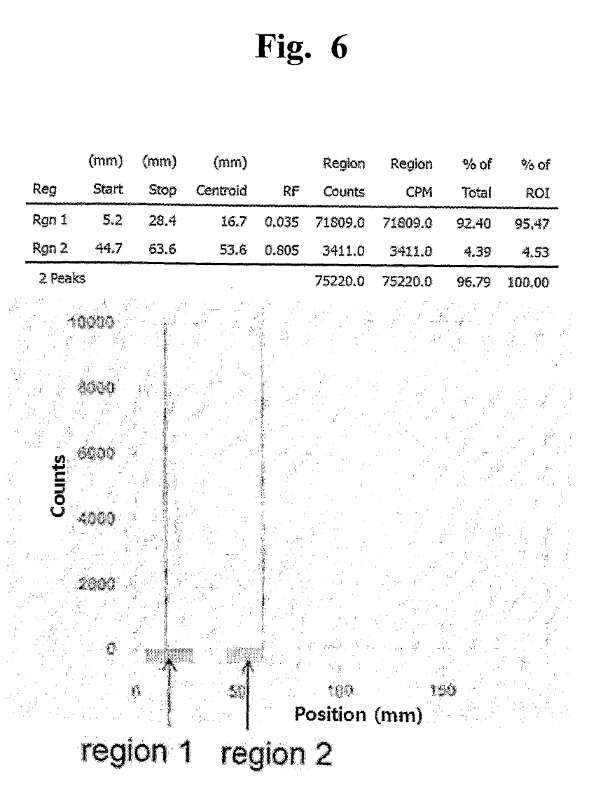

SA-MnFe₂O₄ was radiolabeled with ¹²⁴I (t_{1/2}=4.2 days, β+23%) using IODO-BEADS (Pierce Biochemical Co., USA). 80 μg of MnFe₂O₄ solution and 1 mCi of ¹²⁴I were mixed with activated IODO-BEAD and reacted for 15 min. The ¹²⁴I-SA-Mn Fe₂O₄ was purified from unlabeled free ¹²⁴I by centrifugation (Microcon YM-50, AMICON, USA). Labeling yield was determined by radio-TLC. The radiochemical purity after purification was higher than 92% (FIG. 6).

### Example 18

### PET and MR Imaging of ¹²⁴I-SA-MnFe₂O₄

The ¹²⁴I-SA-MnFe₂O₄ solutions were prepared by dilution to various concentrations (200, 100, 50, 25, 12.5 μM (Mn+Fe), activity: 60, 30, 15, 7.5, 3.8 μCi/mL). In addition, the SA-MnFe₂O₄ and free ¹²⁴I solutions diluted at equal concentrations were prepared. MR and PET imaging of prepared solutions were obtained under the following conditions. MR imaging were performed using a 3D fast Gradient Echo MRI sequence (TR=18.8 ms, TE=5.3 ms, FOV=5×5 cm, matrix=256×256, thickness=3.0 mm, number of experiment=16). Small-animal dedicated microPET (R4 Rodent Model, Concorde Microsystems Inc., USA) was used to obtain dynamic PET imaging for 30 min.

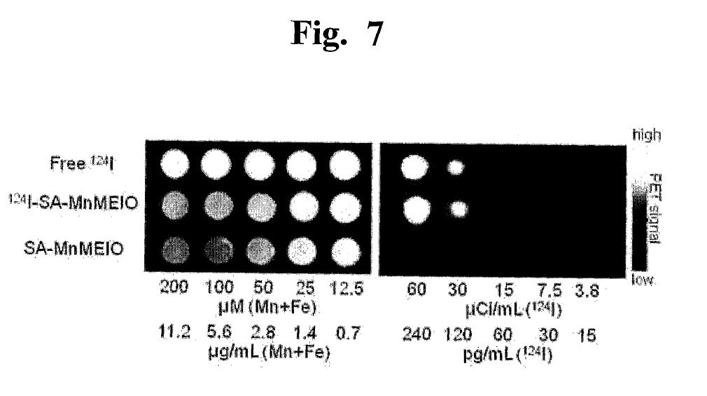

As shown in FIG. 7, PET and MR signals of ¹²⁴I-SA-MnFe₂O₄ were not changed in comparison with MR signal of SA-MnFe₂O₄ and PET signal of free ¹²⁴I solutions although two types of a contrast agent were combined in the present PET/MRI hybrid agent.

### Example 19

### PET Sensitivity of ¹²⁴I-SA-MnFe₂O₄

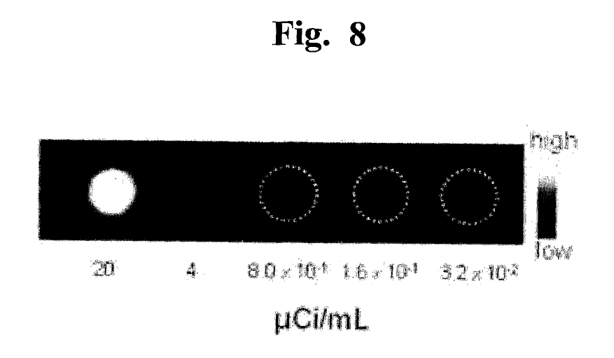

To investigate PET sensitivity of ¹²⁴I-SA-MnFe₂O₄, the solutions diluted to various radioactivities (20, 4, 0.8, 0.16, 0.032 μCi/mL (¹²⁴I)) were prepared and their images were obtained under the same conditions of example 18. In PET imaging, the signals were detected in solutions with radioactivity of up to 4 μCi/mL, but not detected in solution of 0.8 μCi/mL, suggesting that PET detection limit of ¹²⁴I-SA-MnFe₂O₄ has radioactivity in a range of 0.8 to 4 μCi/mL (¹²⁴I) (FIG. 8).

### Example 20

### MR Spatial Resolution of SA-MnFe₂O₄

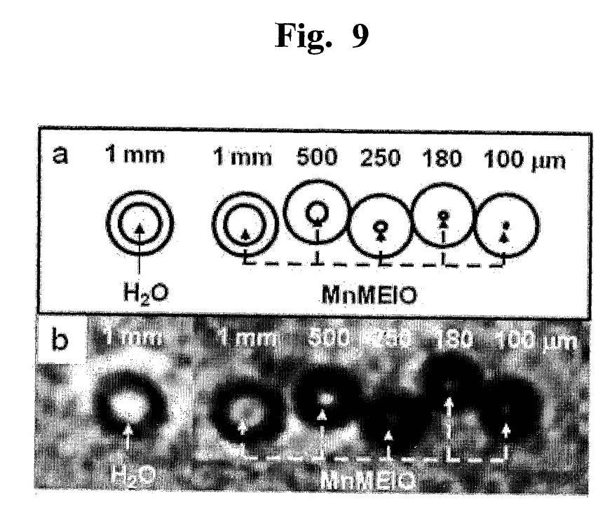

To verify MR spatial resolution of SA-MnFe₂O₄, several tubes with an outer diameter of 1.6 mm and various inner diameters of 1 mm, 500, 250, 180 and 100 μm were arranged and fixed using 1% agarose in phantom. SA-MnFe₂O₄ solution containing Mn+Fe concentration (50 mg/mL) was filled in tubes and tertiary distilled water was filled in the tube with inner diameter of 1 mm as a control. MR images were obtained under the same conditions as Example 18 in 1.5 T. MR images could be distinctly distinguished up to inner diameters of 0.25 mm of the tubes. However, MR signals could not be detected in a distinctly differentiate manner for tubes with inner diameters of below 0.25 mm, due to detection limitations of MR device (FIG. 9).

### Example 21

### PET and MR Imaging of ¹²⁴I-Labeled Magnetic Nanoparticles

SA-coated nanoparticles (SA-MnFe₂O₄, SA-FePt and SA-Fe₃O₄) were radiolabeled with ¹²⁴I using IODO-BEADS. 126 μg SA-MnFe₂O₄, 153 μg SA-FePt and 156 μg SA-Fe₃O₄ solutions were mixed with 214, 103 and 212 μCi ¹²⁴I, respectively. Each mixture was reacted for 15 min with shaking under addition of IODO-BEADS.

MR images of ¹²⁴I-labeled SA-MnFe₂O₄, SA-Fe₃O₄ and SA-FePt were obtained after PET scanning. PET images were collected from signals obtained by OSEM method for 30 min and MR imaging was carried out using a 3D fast Gradient Echo MRI sequence in 1.5 T (TR=8.0 ms, TE=3.2 ms, Flip angle (FA)=20, FOV=10×10 cm, locs per slab=34, matrix=256×256, number of experiment=8).

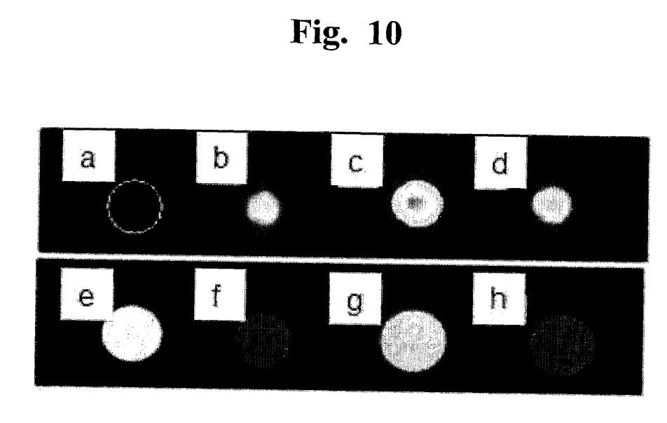

All ¹²⁴I-labeled nanoparticles were observed to show strong PET and MR signals as shown in FIG. 10.

### Example 22

### PET and MR Imaging of Rat Injected with ¹²⁴I-SA-MnFe₂O₄

The reconstituted ¹²⁴I-SA-MnMEIO (80 μg, 110 μCi) in saline (less than 70 μL) was subcutaneously injected into the right front paw of Sprague-Dawley rats (Central Lab. Animal, Inc., Korea, male, 320 g, 12 week-old). Small-animal dedicated microPET (R4 Rodent Model, Concorde Microsystems Inc., USA) was used to obtain dynamic PET imaging for 1 hr. During microPET and MR imaging, rats were anesthetized by inhalation of isoflurane and oxygen mixture. Right after PET scan, MR imaging were performed using a 3D fast Gradient Echo MRI sequence (TR=8.0 ms, TE=3.2 ms, Flip angle (FA)=20, bandwidth=31.25, FOV=10×10 cm, locs per slab=34, matrix=256×256, phase FOV=1, number of experiment=8).

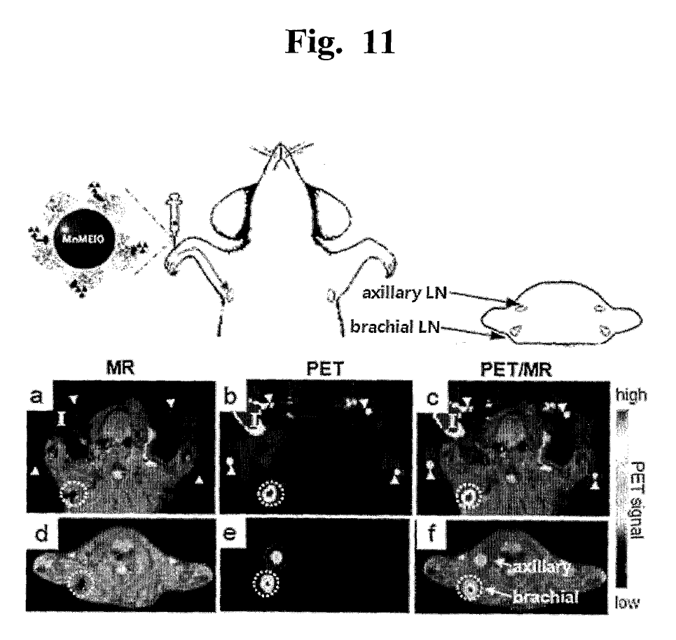

At 1 hr post-injection of ¹²⁴I-SA-MnFe₂O₄ nanoprobes onto the right forepaw, anatomical upper part of rat was observed in detail, identifying several black spots in coronal view of MR image (FIG. 11a). In PET images, each upper and lower spot in two strong red spot is derived from injection site and brachial lymph node (LN, white circle) (FIG. 11b). Although PET is an imaging technique with high sensitivity, it doesn't provide anatomical information. It is only in the case which PET and MR images are completely overlapped in the combined image to provide accurate position of brachial LN (white circle, FIG. 11c) and anatomical shape of rat. In addition, the dual-modality PET/MRI probe of the present invention was detected in the transverse images, and axillary LN also was definitely distinguished from other LNs (FIGS. 11d-11f). In MR image, brachial LN was observed as a strong black spot in lower right part (white circle, FIG. 11d) but axillary LN detected as a blurry black spot was not clearly determined (red circle, FIG. 11d). As a complementary modality technique, the observation of two spots in PET image is very critical (FIG. 11e). By overlapping images from two separate methods, a blue spot in the PET image was definitely matched with the MR detected position, demonstrating the blue spot is the position of an axillary LN, while the stronger red/blue spot coincides with the MR determined position that originated from the brachial LN (FIG. 11f). Interestingly, PET image has the very low background, suggesting that: ¹²⁴I-SA-MnFe₂O₄ dual probe is highly stable in physiological condition; ¹²⁴I do not become detached from the ¹²⁴I-SA-MnFe₂O₄ probes; and intact ¹²⁴I-SA-MnFe₂O₄ is moved along the lymphatic duct.

### Example 23

### Resection of Lymph Node

To verify imaging results described above, brachial LNs from right and left hand sides were dissected and re-examined by PET and MRI. After PET and MR images of rat injected with nanoparticles were taken, brachial lymph nodes on both sides were resected at 40 min post-injection of methylene blue dye. Resected lymph nodes were fixed on 1% agarose gel. PET and MR images were taken as previous conditions.

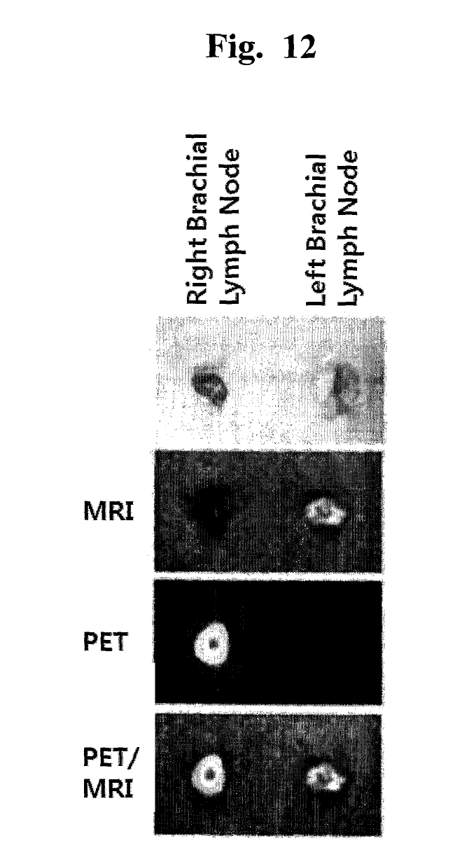

Consistent with in vivo imaging results, PET and MR images of resected lymph nodes exhibited strong PET and MR signals in only the lymph node on the right side compared to the contra-lateral brachial lymph node (FIG. 12).

Having described a preferred embodiment of the present invention, it is to be understood that variants and modifications thereof falling within the spirit of the invention may become apparent to those skilled in this art, and the scope of this invention is to be determined by appended claims and their equivalents.

1. A dual-modality PET (positron emission tomography)/MRI (magnetic resonance imaging) contrast agent, comprising a hybrid nanoparticle which comprises (a) a magnetic signal generating core; (b) a water-soluble multi-functional ligand coated on the signal generating core; and (c) a positron emitting factor linked to the water-soluble multi-functional ligand.

2. The dual-modality PET/MRI contrast agent according to claim 1, wherein the magnetic signal generating core comprises a metal, a metal chalcogen, a metal pnicogen, an alloy or a multi-component hybrid structure thereof.

3. The dual-modality PET/MRI contrast agent according to claim 1, wherein the magnetic signal generating core is a paramagnetic or a superparamagnetic signal generating core.

4. The dual-modality PET/MRI contrast agent according to claim 3, wherein the superparamagnetic signal generating core has a saturation magnetization (Mₛ) value in a range of 20-1000 emu/g.

5. (canceled)

6. The dual-modality PET/MRI contrast agent according to claim 3, wherein the signal generating core has the spin relaxivity coefficient value in a range of 300-1000 mM⁻¹sec⁻¹.

7. The dual-modality PET/MRI contrast agent according to claim 2, wherein the metal nanoparticle comprises transition metals, Lanthanide metals or Actinide metals.

8. The dual-modality PET/MRI contrast agent according to claim 2, wherein the metal chalcogen nanoparticle core comprises a M^{a}ₓA_{y} or M^{a}ₓM^{b}_{y}A_{z} nanoparticle (M^{a} and M^{b} independently represents one or more elements selected from the group consisting of Group 1 metal elements, Group 2 metal elements, transition metal elements, metal and metalloid elements of Groups 13-15 elements, Lanthanide metal elements and Actinide metal elements; A is selected from the group consisting of O, S, Se, Te and Po; 0≦x≦32, 0≦y≦32, 0<z≦8).

9. The dual-modality PET/MRI contrast agent according to claim 2, wherein the metal pnicogen nanoparticle core comprises a M^{c}ₓA_{y} or M^{c}ₓM^{d}_{y}A_{z} nanoparticle (M^{c} and M^{d} independently represents one or more elements selected from the group consisting of Group 1 metal elements, Group 2 metal elements, transition metal elements, metal and metalloid elements of Group 13-14 elements, Lanthanide metal elements and Actinide metal elements; A is selected from the group consisting of N, P, As, Sb and Bi; 0≦x≦40, 0≦y≦40, 0<z≦8).

10. The dual-modality PET/MRI contrast agent according to claim 2, wherein the alloy nanoparticle comprises a M^{e}ₓM^{f}_{y} nanoparticle (M^{e}=one or more elements selected from transition metal elements selected from the group consisting of Ba, Cr, Mn, Fe, Co, Ni and Cu, and Lanthanide metal elements and Actinide metal elements selected from the group consisting of Gd, Tb, Dy, Ho, Sm, Nd and Er; M^{f}=one or more elements selected from the group consisting of Group 1 metal elements, Group 2 metal elements, Group 13 elements, Group 14 elements, Group 15 elements, Group 16 elements, transition metal elements, Lanthanide metal elements and Actinide metal elements; 0<x≦20, 0<y≦20)

11. The dual-modality PET/MRI contrast agent according to claim 2, wherein the magnetic signal generating core comprises:
1) the metal nanoparticle, M (M=Ba, Cr, Mn, Fe, Co, Zn, Nb, Mo, Zr, Te, W, Pd, Gd, Tb, Dy, Ho, Er, Sm or Nd);
2) the alloy nanoparticle, M^{e}ₓM^{f}_{y} (M^{e} and M^{f} independently represent one or more elements selected from the group consisting of Co, Fe, Mn, Ni, Mo, Si, Al, Cu, Pt, Sm, B, Bi, Cu, Sn, Sb, Ga, Ge, Pd, In, Au, Ag and Y; 0<x≦20, 0≦y≦20);
3) the metal oxide nanoparticle, M^{a}ₓO_{y}, in the metal chalcogen nanoparticles (M^{a}=one or more elements selected from the group consisting of Ba, Cr, Co, Fe, Mn, Ni, Cu, Zn, Nb, Pd, Ag, Au, Mo, Si, Al, Pt, Sm, B, Bi, Sn, Sb, Ga, Ge, Pd, In, Gd, Tb, Dy, Ho, Er, Sm and Nd; 0<x≦16, 0≦y≦8); or
4) the multi-component hybrid structure thereof.

12. (canceled)

13. The dual-modality PET/MRI contrast agent according to claim 1, wherein the water-soluble multi-functional ligand comprises an attachment region (L_{I}) linked to the signal generating core.

14. (canceled)

15. The dual-modality PET/MRI contrast agent according to claim 13, wherein the water-soluble multi-functional ligand comprises an active ingredient-binding region (L_{II}) for binding of active ingredients and/or a cross-linking region (L_{III}) for cross-linking between water-soluble multi-functional ligands.

16. The dual-modality PET/MRI contrast agent according to claim 13, wherein the attachment region (L_{I}) comprises a functional group selected from the group consisting of —COOH, —NH₂, —SH, —CONH₂, —PO₃H, —OPO₃H₂, —SO₃H, —OSO₃H, —N₃, —NR₃OH (R=CₙH₂ₙ₊₁, 0≦n≦16), —OH, —SS—, —NO₂, —CHO, —COX (X=F, Cl, Br or I), —COOCO—, —CONH—, —CN and hydrocarbon having at least two carbon atoms.

17. The dual-modality PET/MRI contrast agent according to claim 15, wherein the active ingredient-binding region (L_{II}) comprises one or more functional groups selected from the group consisting of —SH, —COOH, —CHO, —NH₂, —OH, —PO₃H, —OPO₃H₂, —SO₃H, —OSO₃H, —NR₃⁺X⁻ (R=CₙHₘ 0≦n≦16, 0≦m≦34, X=OH, Cl or Br), NR₄⁺X⁻ (R=CₙHₘ, 0≦n≦16, 0≦m≦34, X=OH, Cl or Br), —N₃, —SCOCH₃, —SCN, —NCS, —NCO, —CN, —F, —Cl, —Br, —I, an epoxy group, —ONO₂, —PO(OH)₂, —C═NNH₂, —HC═CH— and —C≡C—.

18. The dual-modality PET/MRI contrast agent according to claim 15, wherein the cross-linking region (L_{III}) comprises one or more functional groups selected from the group consisting of —SH, —COOH, —CHO, —NH₂, —OH, —PO₃H, —OPO₃H₂, —SO₃H, —OSO₃H, Si—OH, Si(MeO)₃, —NR₃⁺X⁻ (R=CₙHₘ, 0≦n≦16, 0≦m≦34, X=OH, Cl or Br), NR₄⁺X⁻ (R=CₙHₘ, 0≦n≦16, 0≦m≦34, X=OH, Cl or Br), —N₃, —SCOCH₃, —SCN, —NCS, —NCO, —CN, —F, —Cl, —Br, —I, an epoxy group, —ONO₂, —PO(OH)₂, —C═NNH₂, —HC═CH— and C≡C—.

19. The dual-modality PET/MRI contrast agent according to claim 1, wherein the water-soluble multi-functional ligand comprises a chemical monomer, a polymer, a protein, a carbohydrate, a peptide, a nucleic acid, a lipid or an amphiphilic ligand.

20-22. (canceled)

23. The dual-modality PET/MRI contrast agent according to claim 1, wherein the water-soluble multi-functional ligand comprises a protein selected from the group consisting of albumins, avidin, antibodies, secondary antibodies, cytochromes, casein, myosin, glycinin, carotene, collagen, globular proteins and light proteins.

24. (canceled)

25. The dual-modality PET/MRI contrast agent according to claim 1, wherein the dual-modality PET/MRI contrast agent is used for cancer imaging.

26. The dual-modality PET/MRI contrast agent according to claim 1, wherein the dual-modality PET/MRI contrast agent is used for imaging of lymphatic system.

27. The dual-modality PET/MRI contrast agent according to claim 26, wherein the dual-modality PET/MRI contrast agent is used for imaging of sentinel lymph node (SLN).
